(19) United States
(12) Patent Application Publication
(10) Pub. No.: US 2024/0390145 A1
(43) Pub. Date: Nov. 28, 2024

(54) SYSTEMS AND METHODS FOR DEPLOYING DEVICES INTO CARDIAC TISSUE

(71) Applicant: Ancora Heart, Inc., Santa Clara, CA (US)

(72) Inventor: Russel M. SAMPSON, Palo Alto, CA (US)

(21) Appl. No.: 18/645,348

(22) Filed: Apr. 24, 2024

Related U.S. Application Data
(60) Provisional application No. 63/497,967, filed on Apr. 24, 2023.

(51) Int. Cl. A61F 2/24; A61L 27/16; A61L 27/52

(52) U.S. Cl. CPC A61F 2/2466; A61F 2/2457; A61L 27/16; A61L 27/52; A61L 2430/20

(57) ABSTRACT

This application generally relates to systems and methods for synchronizing deployment of various devices into cardiac tissue with an ECG waveform. The systems and methods may include use of a pressurized gas to actuate deployment and anchoring of the devices, e.g., implants configured to reshape a heart ventricle, into cardiac tissue. Deployment of the devices may occur during a portion of the cardiac cycle in which myocardial tissue may be at its thickest, e.g., at the end of systole.

### CROSS-REFERENCE TO RELATED APPLICATIONS

This application claims priority to U.S. Provisional Application No. 63/497,967, filed on Apr. 24, 2023, which is hereby incorporated by reference in its entirety.

### FIELD

This application generally relates to systems and methods for synchronizing deployment of various devices into cardiac tissue with an ECG waveform. The devices may be implants configured to reshape a heart ventricle. Deployment of the devices may occur during a portion of the cardiac cycle in which myocardial tissue may be at its thickest, e.g., at the end of systole.

### BACKGROUND

Many systems and methods have been employed in the catheterization lab in order to place devices, e.g., CRT leads, replacement valves, and tissue anchors, into myocardial tissue. In some applications, including dilated cardiomyopathies such as heart failure with reduced ejection fraction (HFrEF), disease progression may lead to reduced wall thickness. Additionally, acute assault from a virus or other agent, or an ischemic event may at times lead to localized thin-walled regions composed of at least some scar tissue. In these environments, placing devices into the myocardium without perforation may be challenging. To overcome this challenge, it may be helpful to recognize that even in regions of ischemia or scar tissue, the myocardium tends to thicken somewhat during systole and become thinner during diastole. Rarely, the reverse can occur, such that the localized thin-walled region thickens during diastole.

In those regions of ischemia or scar tissue that may not thicken at any point throughout the cardiac cycle, ECG synchronization alone may be insufficient to prevent perforation. Under these conditions it may be desirable to locally augment wall thickness, either as a stand-alone process or in combination with ECG synchronization of device deployment. However, navigating around or within the left ventricle (LV) or within the myocardium may be difficult, and navigating back to a specific location a second time can be especially difficult.

Accordingly, it would be beneficial to have new systems and methods that utilize the change in cardiac tissue thickness between systole and diastole, coupled with ECG signals, to deploy devices into thin-walled regions when the regions are at their maximum thickness. It would also be useful to have systems and methods that mitigate the challenge of navigating to a specific location to augment wall thickness, and then returning to that same location to place a device. It would further be useful to have a system and method that provides for real-time wall thickness augmentation and device placement.

### SUMMARY

Described herein are systems configured to synchronize the deployment of various devices into tissue of a patient, e.g., a heart tissue, with an ECG waveform from the patient. In general, the systems may include a deployment catheter, and a controller comprising one or more processors, where the one or more processors may be configured to analyze an ECG signal from a patient and issue (e.g., automatically issue) a control signal to the deployment catheter to deliver the device into one or more target sites of a cardiac tissue. Some variations of the system may also include a guide catheter and/or a multi-window catheter. The one or more target sites may include an area of reduced tissue thickness. In some variations, the area of reduced tissue thickness may be about 4 mm or less. This amount of reduced thickness may be the thickness of the tissue during a diastolic portion of a cardiac cycle. Tissue thickness may be measured using various imaging modalities, e.g., CT, MRI, or ultrasound.

The control signal issued by the one or more processors may be operable to trigger the deployment catheter to deliver the device therefrom during certain portions of the ECG signal. For example, the portion of the ECG signal may correspond to an end systolic portion of a cardiac cycle. Deployment may be synchronized with other portions of the ECG signal that may be associated with the tissue having a greater thickness than portions of the ECG signal that may be associated with tissue having a reduced thickness, e.g., portions of the ECG signal that correspond to a diastolic portion of the cardiac cycle. In some variations, deployment of the devices may be synchronized with the portion of the ECG signal comprising a QRS complex. In other variations, deployment of the devices may be synchronized with the portion of the ECG signal comprising an ST segment.

The deployment catheter of the systems may include an elongate shaft, a handle coupled to a proximal end of the shaft, a slider configured to translate along a longitudinal axis of the shaft, and an actuator coupled to a proximal end of the handle. The actuator may be configured to deliver the device from a lumen of the catheter according to the control signal. In some variations, the actuator may comprise a solenoid valve, e.g., a dual-acting solenoid valve. In other variations, the actuator may further comprise a cylinder coupled to the handle, which may be configured to be filled with a pressurized gas to aid in the deployment of a device into the cardiac tissue. Exemplary pressurized gases may include without limitation, oxygen, helium, nitrous oxide, nitrogen, medical air, carbon dioxide, hydrogen, or mixtures thereof.

A display may also be provided with the systems, which may be configured to show the ECG signal, one or more physiological signals (e.g., heart rate, respiratory rate, blood pressure), an image of an anatomical area of the patient, real-time deployment of a device, or a combination thereof. The display may further include a manual interface operable by a user to switch the one or more processors to a manual mode in which the user may be able to manually control deployment of the device from the deployment catheter. However, in these manual variations, deployment of the device from the deployment catheter and into tissue will still be synchronized with a portion of the ECG signal believed to be associated with a maximum thickness of the tissue.

Some variations of the systems may include an ancillary catheter configured to deliver a substance into the area of reduced tissue thickness to increase its thickness prior to device deployment. The substance may include any biocompatible material or combination of biocompatible materials capable of increasing the thickness of a tissue. For example, biocompatible materials that may be combined or enzymatically or non-enzymatically degraded to form a material capable of increasing tissue thickness may be employed. For example, a combination of fibrinogen and thrombin to form a material comprising fibrin may be used. In other instances, various types of hydrogels may be delivered into the area of reduced thickness. The hydrogels may include natural or synthetic components and/or polymers. For example, a natural hydrogel may include fibrin, collagen, gelatin, or combinations thereof. When a synthetic hydrogel is employed, it may include polyethylene glycol (PEG). Other exemplary hydrogels include without limitation, double network hydrogels. The double network hydrogel may include a first network of poly(ethylene glycol) methyl ether methacrylate and second network comprising acrylic acid. It is understood that other types of hydrogels and substances may be employed.

Methods for synchronizing the deployment of various devices into tissue of a patient, e.g., a heart tissue, with an ECG waveform from the patient are also described herein. In general, the methods may include obtaining an ECG signal from a patient, synchronizing deployment of the device, from a deployment catheter with a portion of the ECG signal, and delivering the device into one or more target sites of a cardiac tissue. As mentioned above, the one or more target sites may include an area of reduced tissue thickness. In some variations, the area of reduced thickness may be about 4 mm or less. This amount of reduced thickness may be the thickness of the tissue during a diastolic portion of a cardiac cycle.

Deployment of a device may be synchronized with a portion of the ECG signal. In some variations, the portion of the ECG signal corresponds to an end systolic portion of a cardiac cycle. In other variations, the portion of the ECG signal corresponds to a QRS complex of the ECG signal. In further variations, deployment of a device may be synchronized with an ST segment of the ECG signal. Synchronizing deployment may be accomplished using one or more processors configured to analyze the ECG signal and then automatically issue a control signal to the deployment catheter that may trigger deployment of the device. In some variations of the systems and methods, the one or more processors may obtain and analyze an ECG signal, but may also include a feature or processing step, respectively, whereby deployment of the device may be at least partially manually controlled by a user instead of automatically controlled. In some instances, delivering a device into one or more target sites comprises use of a pressurized gas. The pressurized gas may include oxygen, helium, nitrous oxide, nitrogen, medical air, carbon dioxide, hydrogen, or mixtures thereof. The methods described herein may further include showing on a display of the system, an ECG signal, one or more physiological signals, an image of an anatomical area of the patient, real-time deployment of a device, or a combination thereof.

In some variations, the methods may include injecting a substance into the area of reduced tissue thickness to increase its thickness. Injecting the substance may typically occur prior to deployment of the device, but in some instances may occur simultaneously with device deployment. Once within the one or more target sites and/or target area of tissue, the injected substance may increase the thickness of the tissue at the one or more target sites and/or target area to at least about 5 mm (measurement taken during diastole). As previously mentioned, the injected substance may include any biocompatible material capable of increasing the thickness of a tissue. For example, a hydrogel may be delivered into the area of reduced thickness. Exemplary hydrogels include without limitation, double network hydrogels. The double network hydrogel may include a first network of poly(ethylene glycol) methyl ether methacrylate and second network comprising acrylic acid. It is understood that other types of hydrogels and substances may be injected into the tissue.

Various devices may be deployed using the systems and methods described herein. The devices may be pre-loaded within a lumen of the deployment catheter. In one variation, the devices may include an implant configured to reshape a heart ventricle, e.g., a left ventricle (LV). Some variations of the implant may include a plurality of anchors coupled to a tether, where the specific device component deployed into tissue is one or more of the anchors. In these variations, the implant may further include a plurality of force distribution members. Each force distribution member of the plurality of force distribution members may be coupled to the tether between two anchors. The systems and methods described herein may be configured to deploy the devices within various cardiac tissues and/or cardiovascular tissues. For example, the devices (e.g., anchors) may be deployed within an atrial tissue, a ventricular tissue such as left ventricular (LV) tissue, or at least a portion of a heart valve such as an aortic valve, a mitral valve, a tricuspid valve, and a pulmonary valve. The cardiac tissue in some instances may include the myocardium of the heart.

### BRIEF DESCRIPTION OF THE DRAWINGS

FIG. 1A is a schematic representation of a region of tissue (1) at its end diastolic position, with a localized thin region (2), with a thickness (3).

FIG. 1B is a schematic representation of the same region of tissue in FIG. 1A, but at its end systolic position, with localized thin region (2) and a greater thickness (4).

FIG. 2A is a schematic short axis (SAX) view of an exemplary left ventricle (LV), with an LV chamber (5), left ventricular outflow tract (6), endocardium (7), myocardium (8), endocardium at end diastole (9a), endocardium at end systole (9b), and a localized thin-wall region (10).

FIG. 2B is a schematic representation illustrating access into the LV using an exemplary guide catheter (11) with a distal tip (12).

FIG. 2C is a schematic representation of an exemplary multi-window catheter (13) with multiple windows (14) advanced through the guide catheter (11) of FIG. 2B and out distal end (12) to track around endocardium (9a) and span a substantial portion of the LV.

FIG. 2D is a schematic representation of an exemplary delivery catheter (15) having a distal tip (16) and containing a device for deployment (17) placed within the multi-window catheter (13) of FIG. 2C. The distal tip (16) of the delivery catheter (15) may be advanced through the multi-window catheter (13) and out a distal window (14) of the multi-window catheter (13). Device (17) is shown located within myocardium (8) after deployment.

FIG. 2E is a schematic representation of the delivery catheter (15) of FIG. 2D having a distal tip (16) and containing device for deployment (17) advanced through multi-window catheter (13) and out a distal window (14) near thin wall region (10). Device (17) is shown located within myocardium (8) and extending out past pericardium (7) after deployment, representing an exemplary perforation condition.

FIG. 3 depicts an exemplary cardiac ECG signal including a QRS complex (18a), which represents ventricular depolarization and systole, and a T wave (18b), which represents ventricular repolarization and diastole.

FIG. 4 is a schematic diagram of an exemplary system that may be utilized to control deployment of a device. The exemplary system (40) may include an ECG monitor (19), a controller (20), a deployment catheter (21) comprising a handle (22), a release button (23), an actuator (24), an elongate shaft (25) with a distal tip (26), and a device to be deployed (27), as shown in expanded view (28).

FIG. 5 is a schematic representation of the system of FIG. 4 in use in the LV of FIG. 2A.

FIG. 6A is a schematic representation of an exemplary substance catheter (29) having a distal end (30) and a cannula (31) advanced though multi-window catheter (13) and out a window (14) of the multi-window catheter (13) near thin wall region (10), such that cannula (31) is placed within myocardium (8) near thin wall region (10).

FIG. 6B is schematic representation of an exemplary substance (32), which may be radiopaque, delivered through cannula (31) into myocardium (8), near the thin wall region (10) of FIG. 6A, resulting in an augmented wall region (33).

FIG. 6C is a schematic representation of a delivery catheter (15) exiting window (14) of a multi-window catheter (13) to deliver a device (17) into myocardium (8) in the region of augmented wall thickness (33) formed as illustrated in FIG. 6B.

FIG. 7A is a schematic representation of an exemplary fluoroscopic image of a catheter (29) having a distal end (30), and cannula (31) exiting a multi-window catheter (13) and into myocardium (8) (not radiopaque). A radiopaque indicator wire (34) may be utilized to determine the location of endocardium (9) (not radiopaque).

FIG. 7B depicts the fluoroscopic image of FIG. 7A, with the radiopaque indicator wire (34) bent to indicate a depth of penetration beyond endocardium (9) (not radiopaque), and a substance (32) delivered through cannula (31).

FIG. 8A is a schematic representation of a single bolus of substance (32) injected through cannula (31) to locally augment a narrow thin region (35) of tissue (1) according to one variation.

FIG. 8B is a schematic representation of bolus (32) injected at several discrete locations to augment a wider region (36) of tissue (1) according to one variation.

FIG. 8C is a schematic representation of bolus (32) stacked along several locations to create a thicker region (37) of tissue (1) according to one variation.

FIG. 8D is a schematic representation of an exemplary extended thin region (38) of tissue (1), with a thickness (39).

FIG. 8E is a schematic representation of bolus (32) injected at several discrete locations spanning the width of the extended thin wall region (38) shown in FIG. 8D to locally augment its thickness, while thickness (39) persists at locations between bolus injections (32).

FIGS. 9A and 9B depict side perspective views of an exemplary handle (22) of the deployment catheter (21) shown in FIG. 4. FIG. 9A shows the handle (22) in the stowed configuration, and FIG. 9B shows the handle (22) in an actuated configuration.

FIG. 10 depicts an exemplary implant including a plurality of devices (anchors) coupled to a tether.

FIG. 11 depicts an exemplary device configured to embed into cardiac tissue.

FIGS. 12A and 12B depict an exemplary multi-window catheter of the system. FIG. 12A provides a perspective view of a distal portion of the multi-window catheter, and FIG. 12B provides a cross-sectional view of the multi-window catheter shown in FIG. 12A.

FIG. 13 is a graph that illustrates the relationship between left ventricular volume and different portions of the ECG signal.

### DETAILED DESCRIPTION

Described herein are systems and methods for synchronizing the deployment of various devices into tissue of a patient, e.g., a heart tissue, with an ECG waveform from the patient. The patients may have a medical condition, e.g., heart failure, that results in enlargement of one or more atria or ventricles of the heart, and which in turn may result in thinning of the myocardium. Deployment of devices that improve cardiac output in these patients may be difficult due to the risk of perforating the thin myocardium. Accordingly, the systems and methods described herein may reduce the risk of myocardial perforation (and other types of tissue perforation) by automatically triggering device deployment at certain portions of the cardiac cycle when the myocardium (or other tissue) may be thicker.

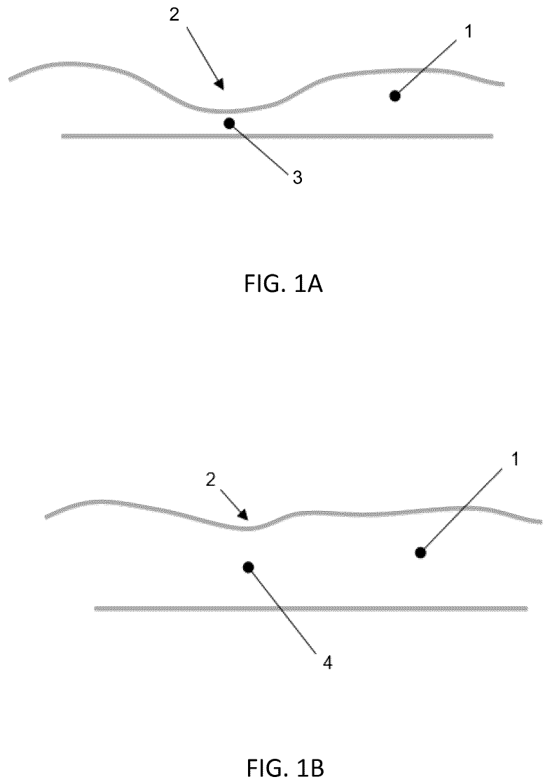

Referring to FIGS. 1A and 1B, the difference in tissue thickness is illustrated between cardiac tissue at the end of diastolic and systolic portions of the cardiac cycle. For example, in FIG. 1A, a region of tissue (1) is shown at the end of diastole having a localized thin region (2) with a first thickness (3). FIG. 1B shows the same region of tissue in FIG. 1A, but at the end of systole. In FIG. 1B, the localized thin region (2) has a second, greater thickness (4) than the first thickness.

### Systems

The systems described herein may generally include a deployment catheter, and a controller comprising one or more processors, where the one or more processors may be configured to analyze an ECG signal from a patient and issue a control signal to the deployment catheter to deliver the device into one or more target sites of a cardiac tissue. The one or more target sites may include an area of reduced tissue thickness, e.g., a thickness of about 4 mm or less during a diastolic portion of a cardiac cycle. Tissue thickness may be measured using imaging modalities such as, but not limited to, CT, MRI, or ultrasound. In some variations, the systems may also include a guide catheter, a multi-window catheter configured such that the multiple windows may help guide device deployment, and an ancillary catheter configured to inject a substance into the one or more target sites and/or tissue area having reduced tissue thickness.

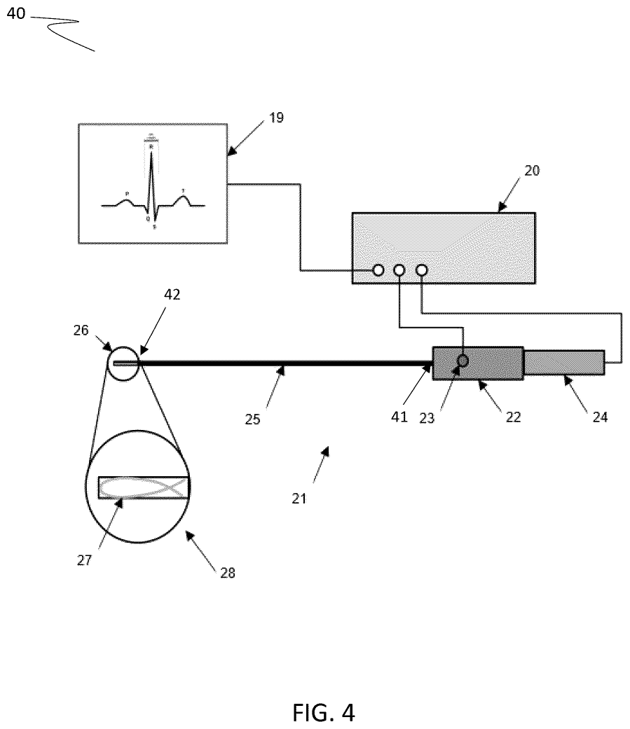

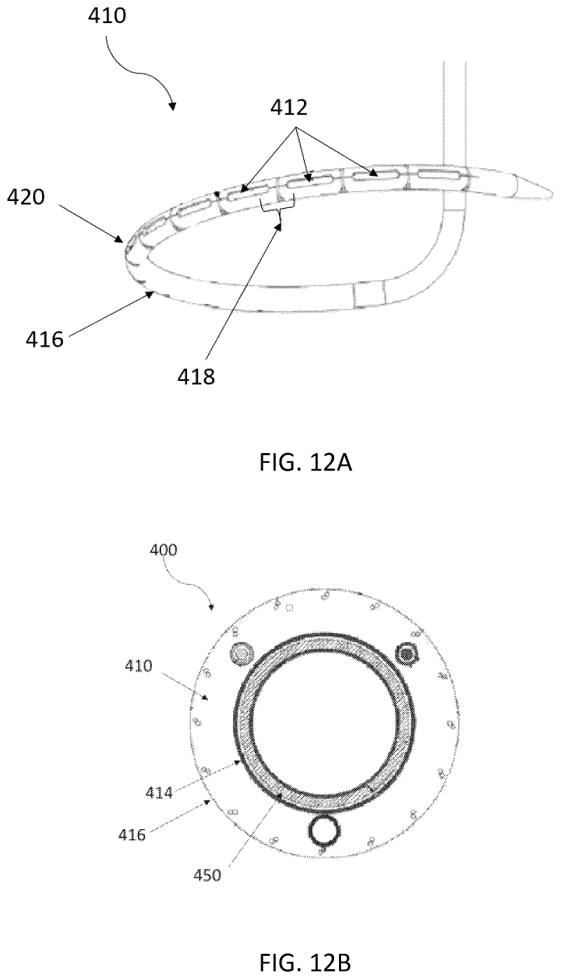

Referring to FIG. 4, an exemplary system for synchronizing deployment of a device into cardiac tissue of a patient with an ECG signal is provided. In FIG. 4, the system (40) may include a display or an ECG monitor (19) electrically connected to a controller (20), which may include one or more processors configured to obtain and analyze the ECG signal (e.g., an ECG waveform). A deployment catheter (21) having a proximal end (41) and a distal end (42) may also be connected to the controller (20). In use, an operator/user may advance an elongate shaft (25) of the deployment catheter (21) through a multi-window catheter (shown in FIG. 12 and described in further detail below) until a tip of the deployment catheter is adjacent a target site of the cardiac tissue. The operator/user may then grasp the catheter (21) by a handle (22) attached to the proximal end (41) of the elongate shaft (25), to advance the deployment catheter (21) to the proper location against or into the myocardium. Once the desired position of the deployment catheter (21) has been achieved and normal sinus rhythm is detected in the patient, the operator/user may activate (e.g., press) a button comprising, e.g., a switch (e.g., a 12V switch (23)), which may send an enabling signal to the controller (20), such that upon receiving the next QRS signal from the patient, the controller (20) may send a control signal to an actuator (24) of the handle (22). Actuation of the actuator (24), as further described below, may then trigger deployment of a device, e.g., anchor (27), depicted in expanded view (28), through a distal tip (26) of the elongate shaft (25) and into the target site of the myocardium during a target portion of the ECG signal.

### Deployment Catheters

The deployment catheters included as part of the systems may generally include an elongate shaft, a handle coupled to a proximal end of the shaft, and a slider configured to translate along a longitudinal axis of the shaft. The handle may include an actuator, which may be coupled to any portion thereof, e.g., a proximal end of the handle, a distal end of the handle, above or below the handle, and be configured to deliver the device from a lumen of the deployment catheter according to the control signal. In some variations, the actuator may comprise a solenoid valve, e.g., a dual-acting solenoid valve. In other variations, the actuator may further comprise a cylinder coupled to the handle, which may be configured to be filled with a pressurized gas to aid in the deployment of a device into the cardiac tissue. Exemplary pressurized gases may include without limitation, oxygen, helium, nitrous oxide, nitrogen, medical air, carbon dioxide, hydrogen, or mixtures thereof.

The elongate shaft may be flexible and include a proximal end, a distal end, a longitudinal axis, and a lumen extending along the longitudinal axis between the proximal and distal ends. The elongate shaft may have any suitable length that allows for deployment of a device into the one or more target sites of a tissue. For example, when the device is to be deployed into cardiac tissue such as the left ventricle (LV), the elongate shaft may have a length ranging from about 90 cm to about 110 cm, including all values and sub-ranges therein. For example, the length of the elongate shaft may be about 90 cm, about 95 cm, 100 cm, about 105 cm, or about 110 cm. Additionally, the lumen of the elongate shaft may have any diameter suitable for advancement of devices, e.g., implant delivery catheters, multi-window catheters, and/or ancillary catheters (e.g., substance delivery catheters) therein. In some variations, the size of the elongate catheter may range from 6 Fr to 8 Fr. The elongate shaft may be made from materials such as polyamide, polyurethane, polytetrafluoroethylene, and combinations thereof.

The deployment devices may also include a handle including a body, a proximal pivot attached to the handle body, and a slider including a distal pivot. The slider may be configured to move back and forth along the longitudinal axis of the elongate shaft. An actuator including a cylinder may be coupled to the body of the handle at the proximal pivot and the distal pivot via a proximal axle and a distal axle, respectively. The cylinder may include an inner shaft disposed at least partially within its interior that exits a distal end of the cylinder to attach to the distal axle. With this configuration, when a gas is released into the cylinder, the gas pressure may push at least a portion of the inner shaft out of the distal cylinder end, which in turn may advance the slider distally along the catheter. The slider may be coupled to a push tube of an implant device having a plurality of anchors such that movement of the slider in a distal direction may advance the push tube distally to deploy the anchors into tissue.

The handle and slider may be made from various materials. In general, the materials may be polymeric materials. The polymeric materials may include, but are not limited to, polycarbonate (PC), acrylonitrile butadiene styrene (ABS), polybutylene terephthalate (PBT), polyethylene terepthalate (PET), polypropylene, polyvinyl chloride (PVC), polymethyl methacrylate (PMMA), polyoxymethylene, and polyurethane.

The actuator of the deployment devices may include one or more of a solenoid valve and a cylinder. In some variations, the actuator includes both a solenoid valve and a cylinder. In one variation, the solenoid valve is a double acting (dual acting) solenoid valve. In some variations, the double acting solenoid valve may be coupled to a cylinder of the handle and a switch, e.g., a 12V switch. The default state of the switch may be an “off” state. The cylinder may be a pneumatic tube configured to fill with a pressurized gas when the switch is in an “on” state, which may be a state that allows current to flow to the double acting solenoid valve. The switch may turn to an “on” state during a specific portion of the ECG signal, e.g., that portion of the ECG signal corresponding to the end of systole.

The pressurized gas may comprise oxygen, nitrous oxide, nitrogen, medical air, helium, carbon dioxide, hydrogen, or mixtures thereof. Delivery of a gas to the cylinder of the deployment catheter may occur at pressures sufficient to deploy the devices (e.g., anchors) into tissue, and may range from about 50 psi to about 200 psi, including all values and sub-ranges therein. For example, the gas may delivered at a pressure of about 50 psi, about 55 psi, about 60 psi, about 65 psi, about 70 psi, about 75 psi, about 80 psi, about 85 psi, about 90 psi, about 95 psi, about 100 psi, 105 psi, about 110 psi, about 115 psi, about 120 psi, about 125 psi, about 130 psi, about 135 psi, about 140 psi, about 145 psi, about 150 psi, about 155 psi, about 160 psi, about 165 psi, about 170 psi, about 175 psi, about 180 psi, about 185 psi, about 190 psi, about 195 psi, or about 200 psi. In one variation, the gas is delivered to the cylinder of the deployment catheter at about 100 psi. In other variations, the gas is delivered to the cylinder of the deployment catheter at a pressure of at least about 100 psi.

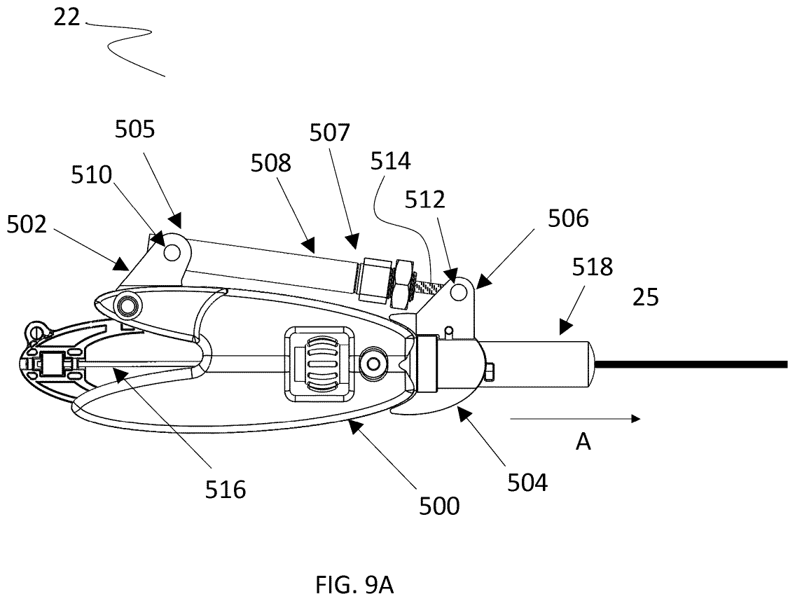

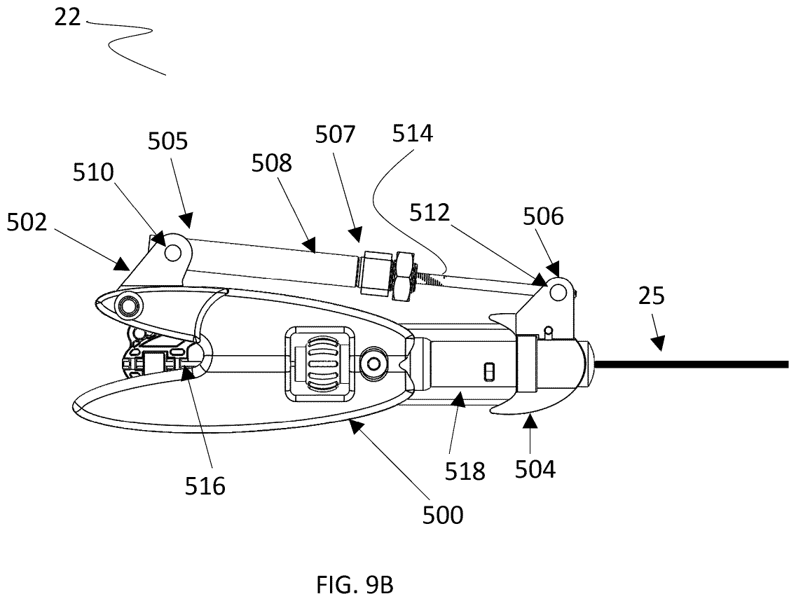

The handle of the deployment catheter illustrated in FIG. 4 may be configured as shown in FIGS. 9A and 9B. In FIG. 9A, the handle (22) is depicted in a stowed configuration, prior to deployment of a device, and in FIG. 9B, the handle (22) is depicted in an actuated configuration, after deployment of the device. The device may comprise one or more anchors configured to embed within cardiac tissue. In some variations, the device may be an implant comprising a plurality of anchors coupled to a tether, where the plurality of anchors may be configured to embed within cardiac tissue upon deployment. The handle (22) may include a body (500) and a proximal pivot (502). A slider (504) including a distal pivot (506) may slidingly engage with the body (500). The slider (504) may be configured to move back and forth along the longitudinal axis of the elongate shaft (25). An actuator including a cylinder (508) having a proximal end (505) and a distal end (507) may be coupled (e.g., rotationally coupled) to the body (500) of the handle (22) at the proximal pivot (502) and the distal pivot (506) via a proximal axle (510) and a distal axle (512), respectively. An inner shaft (514) having a proximal end and a distal end may be at least partially disposed within the cylinder (508) and exit the cylinder distal end (507) to attach to the distal axle (512). When deployment of the device is triggered, as further described below, the handle (22) may transition from a stowed configuration (FIG. 9A) to an actuated configuration (FIG. 9B). The transition from the stowed to the actuated configuration may be accomplished by releasing a pressurized gas into the cylinder (508). The gas pressure may advance at least a portion of the inner shaft (514) out of the cylinder distal end (507). Given that the inner shaft (514) may be coupled at its distal end to the distal pivot (506) of the slider (504) via the distal axle (512), advancing the inner shaft (514) out of the cylinder (508) may advance the slider (504) distally along the shaft (25) in the direction of arrow A. The slider (504) may also be attached to a push tube (516) of an implant delivery catheter such that movement of the slider (504) in a distal direction in turn advances the push tube (516) distally, also in the direction of arrow A, to deploy, e.g., the anchors of an implantable device into tissue.

### Controllers/Processors-Trigger Mechanism

The controller of the systems described herein, e.g., controller (20) shown in FIG. 4, may include one or more processors. The one or more processors may be configured to analyze, e.g., using a signal analyzer unit, an ECG signal from a patient and automatically issue a control signal to the deployment catheter to deliver a device into one or more target sites of a cardiac tissue. The one or more target sites may include an area of reduced tissue thickness, e.g., an area of thickness of about 4 mm or less during a diastolic portion of a cardiac cycle.

The control signal issued by the one or more processors may be operable to trigger the deployment catheter to deliver the device therefrom during a target portion of the ECG signal. For example, the target portion of the ECG signal may correspond to an end systolic portion of a cardiac cycle. Triggering of deployment may be synchronized with other portions of the ECG signal that may be associated with the tissue having a greater thickness than portions of the ECG signal that may be associated with tissue having a reduced thickness, e.g., portions of the ECG signal that correspond to a diastolic portion of the cardiac cycle. In some variations, triggering of deployment of the devices may be synchronized with the portion of the ECG signal comprising a QRS complex. In other variations, triggering deployment of the devices may be synchronized with the portion of the ECG signal comprising an ST segment, or a peak or falling edge of a T wave.

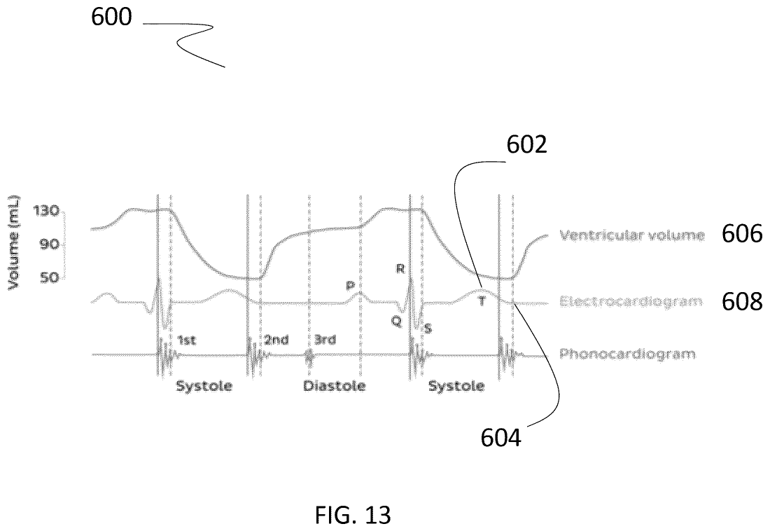

In some variations, the one or more processors may be configured to embed a device (e.g., anchors), in the heart tissue at or close to the end of systole (e.g., the end of ventricular systole). In one variation, the one or more processors may use the falling edge of the T wave as the point at which to embed anchors into the tissue. The start of ventricular systole occurs with depolarization, which generally occurs at the peak of the QRS complex. The end of systole occurs at approximately the peak or falling edge of the T wave. Since it may take some time (e.g., about 60 msec) for the triggering mechanism (e.g., the actuator) to deploy the anchors, the control signal may be issued at a point between the end of the QRS complex and the T wave, and in some instances, well before the end of the latter. The exact timing may depend on what is normal for the patient undergoing the procedure. In some variations, the system may be configured to trigger deployment of a device (e.g., anchors) as follows:
(1) Detect normal sinus rhythm in a patient using one or more processors of the system since the system may generally be configured to only deploy anchors at the end of a normal sinus beat. In this instance, the system (e.g., the one or more processors of the system) may be configured to obtain the following information:
   (a) the shape and width of a normal QRS complex;
   (b) the shape and timing (i.e., the width and position relative to the R wave) of the P wave (if detectable);
   (c) the time between the peak of the R wave and the intended anchor deployment time at or near the end of the T wave; and
   (d) the variability in the timing of the ECG for the patient, to estimate the expected range of timing.
(2) After normal sinus rhythm is detected, a user (e.g., a physician) may press a button that is configured to place the system in an activated state so that it is ready to fire at the appropriate time after the next normal QRS complex is detected. Upon detection of the next normal QRS complex, a control signal may be issued such that one or more anchors are deployed into tissue at the portion of the ECG signal (e.g., at the end of systole, between the peak of the T wave and the falling edge of the T wave) where cardiac tissue such as myocardium may be at its thickest, e.g., at least about 5 mm. For example, referring to FIG. 13, a graph illustrating left ventricular volume at various parts of ECG waveform is provided. In FIG. 13, graph 600 shows that at the end of systole, the part of the ECG waveform (608) corresponding to low ventricular volume (line 606) is between the peak (602) of the T wave to about before the T wave hits the isoelectric baseline (604). Thus, it may be useful to time triggering of the actuator such that the one or more anchors or other devices may be deployed into cardiac tissue between the peak (602) of the T wave and before the T wave returns to the isoelectric baseline (604).

As previously stated, the one or more processors may use the falling edge of the T wave as the point at which to trigger the device to be deployed and embed/anchor into the tissue. In some variations, it may be beneficial for device anchoring to occur when the T wave returns to within about 20% of the T wave peak amplitude from the isoelectric line (baseline).

When analyzing ECG signals, it may also be useful for the one or more processors to be configured to detect and reject abnormal heart beats so that only ECGs exhibiting normal sinus rhythm (NSR) are used when synchronizing deployment of the devices. Exemplary ectopic beats may include premature ventricular contractions (PVCs) and premature atrial contractions (PACs).

In some variations, the one or more processors of the system may be configured to reject abnormal heart beats in which the QRS complex occurs outside of the typical time window exhibited during NSR. In other variations, the ECG signal for a particular patient may first be characterized according to a technique similar to that used for high-resolution signal-averaged electrocardiography. Using this technique, a template for normal beats (e.g., NSR beats) for that patient may be created consisting of three parts: (i) the QRS complex, (ii) the P wave, and (iii) the T wave. In addition, the one or more processors may measure and record the mean and standard distribution of (iv) the PR interval and (v) the RT interval. For example, the QRS complex may first be detected, and (if it correlates well with the previous template (i.e., the “normal” template)), incorporate that new QRS complex into the template (i). As a next step, the P wave may be detected by looking backward in time and incorporating that new P wave into the template (ii). Once the P wave has been identified, the time between the peak of the P and the peak of the R wave may be measured and factored into the measurements of the PR interval. The process may be performed in the same manner when looking forward for the T wave. Additionally, between each adjacent pair of normal beats, the RR interval may be measured, and the average and standard deviation for the RR interval data computed and recorded.

Once the three waveform templates (P, QRS, and T), two pairs of interval data (PR and RT), and the RR interval mean and standard deviation from the patient have been created, the one or more processors may compare them to the ECG signal obtained from the patient during the device deployment procedure in order to detect an abnormal heart beat. For each new heart beat, the one or more processors may reject the heart beat from consideration for triggering the actuator if any one or more of the following are true: 1) the QRS complex for the new beat does not correlate well with the most recent QRS template; 2) the P wave does not correlate well with the most recent P template; and 3) the PR interval for the new beat is more than 1 SD (standard deviation) from the mean for the set of heart beats used in the template.

### Devices

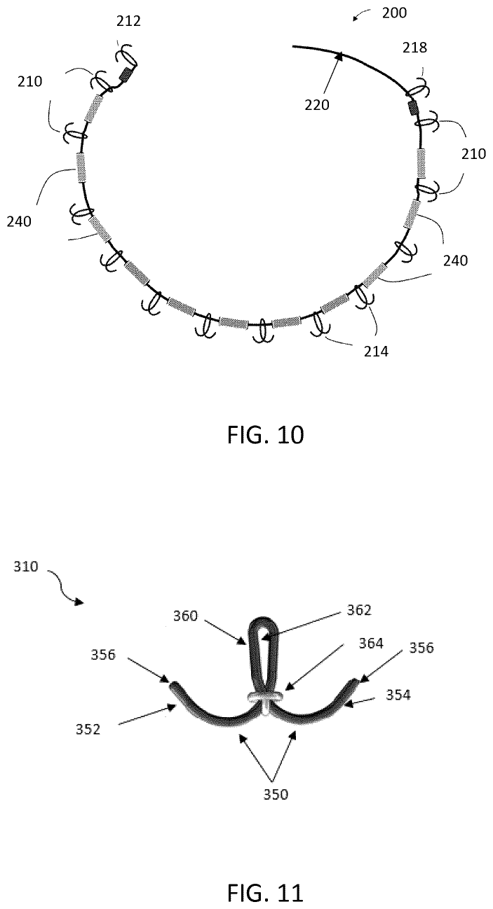

One variation of a device that may be deployed using the systems and methods described herein may be an implant including a plurality of anchors coupled to a tether. In this variation, the device may be configured to secure to a ventricular wall for reshaping a heart ventricle (e.g., reverse-remodeling the ventricle in order to reverse the effect of pathological cardiac remodeling). For example, referring to FIG. 10, an implantable device (200) may comprise a plurality of tethered anchors (210) is shown. The implantable device (200) may comprise a first distal-most anchor (212), one or more secondary anchors (214), and proximal-most terminal anchor (218). The first anchor (212) may be fixedly attached (e.g., knotted, adhered, welded, etc.) to the tether (220). The plurality of secondary anchors (214) may be slidably coupled to the tether (220). For example, the tether may be threaded through an opening in each secondary anchor (214). The terminal anchor (218) may also be slidably disposed about the tether (220). In another variation, the terminal anchor (218) may also be fixedly attached to the tether (220). For example, the tether may be fixedly attached (e.g., knotted, adhered, welded, etc.) to the proximal-most terminal anchor (218). Each anchor (210) may be configured to attach to a portion of the ventricle wall. For example, a portion of each anchor may pierce through and penetrate the tissue of the ventricle wall to secure the implantable device (200) to the ventricle. Force distribution members (FDMs) (240) may be disposed about the tether (220) between or adjacent to all or a subset of the anchors (210). For example, one FDM (240) may be located between each set of two sequential anchors (210). The implantable device may further comprise a lock member, which may operate to secure the implantable device in a cinched configuration, as further discussed below.

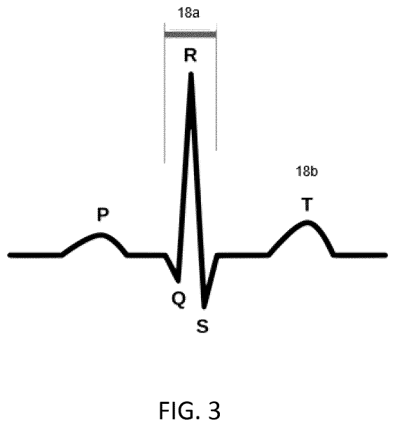

In some variations, each anchor of the plurality of anchors may comprise a tissue-attachment structure and a tether-coupling structure. FIG. 11 depicts one variation of an anchor (310) of an implantable device that may be configured to be secured into the ventricle wall. The anchor (310) may comprise a tissue attachment portion (350) and an eyelet or loop portion (360) that is configured to retain a tether. The tissue attachment portion (350) may be configured to secure the anchor (310) to the ventricle wall, and the eyelet portion (360) may comprise an opening configured to house a tether. For example, as depicted in FIG. 3, the tissue attachment portion (350) may comprise a first leg (352) and a second leg (354), each leg having a tissue-piercing end (356) for penetrating cardiac tissue (e.g., piercing through the surface of myocardium), and one or more curves along the length of each leg to engage cardiac tissue. The eyelet portion (360) may be a loop with a central opening (362), such that the tether may be threaded through the opening (362). The anchor may be made of a single, continuous wire (e.g., of nitinol) that extends in a single-turning direction from one end to the other end (e.g., ends (356)), forming a loop (e.g., eyelet (362)) in between the ends. In other variations, an anchor may comprise multiple components secured together, such as by welding, adhesive, or any other suitable methods. For example, the eyelet portion and tissue attachment portion may be comprised of two or more separate wire segments that are secured together. Optionally, the anchor (310) may also comprise a ring-shaped wire, suture or collar (364) that is located at the base of the eyelet or loop (360). The collar (364) creates a closed loop in the eyelet to prevent the anchor from detaching from the tether, and may help to secure the eyelet and/or reinforce the size and shape of the eyelet portion (360). Other anchor variations may not have a collar. The eyelet or loop (360) may have any suitable shape. For example, the eyelet portion (360) may have an elongate shape and/or a narrow profile that tapers to the base, which may facilitate tissue penetration. Alternatively, some anchors may not comprise two legs and an eyelet between them. For example, an anchor may comprise one hook and one loop, having an S-shaped configuration. Alternatively or additionally, an anchor may comprise a plurality of struts attached to a loop. Devices described herein may comprise any suitable tissue attachment devices such as clips, clamps, springs, hooks, sutures, etc.

The anchor may be comprised of any conformable and/or elastic material. For example, the anchor (either or both the tissue attachment portion and the eyelet or loop portion) may be made of an elastic material (e.g., a super-elastic material) and/or a shape-memory material. Examples of such materials may include any metals, alloys, such as nickel titanium alloy (Nitinol), or polymers (e.g., rubber, poly-ether ether ketone (PEEK), polyester, nylon, etc.). The anchor may also comprise more than one material. For example, in some variations, the tissue attachment/penetration portion and eyelet portion of the anchor may be comprised of nitinol, and the collar of the anchor may be comprised of polyester. In some variations, the anchor or the collar or both may comprise a radiopaque material. This may provide visibility of the anchors while they are being secured to the ventricle, which may aid the implantation of the device. In some variations, portions of the FDMs may comprise a radiopaque material. Using one or more radiopaque materials in the anchors and/or FDMs may permit fluoroscopic images of the devices to be acquired during implantation, which may facilitate the placement of the implant at the desired locations and with the desired orientation.

Turning back to FIG. 10, the implantable device (200) may further comprise one or more force distribution members (FDMs) (240) slidably coupled to the tether and situated between the plurality of anchors (210), as depicted in FIG. 10. Each FDM (240) may be situated between two anchors (210). In some variations of the implantable device, a FDM (240) may be situated adjacent and distal to the first anchor (212). Similarly, a FDM may be situated adjacent to and proximal to the terminal anchor (218). Any suitable number of FDMs may be positioned between or adjacent to anchors. For example, in some variations, two or more FDMs may be positioned between two anchors. Different numbers of FDMs may be used at different positions of the device. For example, two FDMs may be positioned between the terminal anchors and next-to-terminal anchors (e.g., between the distal-most terminal anchor and the next-to-distal-most anchor, and/or between the proximal-most terminal anchor and a next-to-proximal-most anchor), while one FDM may be positioned between all other sets of anchors. In another variation, one FDM may be positioned between the terminal anchors and next-to-terminal anchors, while two FDMs may be positioned between all other sets of anchors. It is not necessary that the distribution of FDMs be uniform, and any number of FDMs may be positioned adjacent to any anchor. For example, one FDM may be positioned between the proximal-most terminal anchor and a next-to-proximal-most anchor, and subsequent FDMs may be positioned between every-other set of anchors. In one variation, the FDMs (240) may all be the same length. However, in another variation, the FDMs may have varying length. For example, the FDMs near the center of the implantable device may be longer or shorter in length than the FDMs of at the ends of the implantable device. In one variation, the FDMs between the terminal anchors and the next-to-terminal anchors (e.g., between the distal-most terminal anchor and the next-to-distal-most anchor, and/or between the proximal-most terminal anchor and a next-to-proximal-most anchor) may be shorter than the FDMs between the intermediate anchors (e.g., the anchors in a central region of the implant). However, the FDMs may be of any suitable length.

Further, the FDMs (240) may have a lumen configured to house the tether (220). Thus, the FDM may be slidably disposed about the tether by extending the tether though the lumen of the FDM. In the variation depicted in FIG. 10, the FDMs (240) have a tubular configuration. However, the FDMs (240) may be of any suitable shape. For example, the FDMs may comprise a rectangular, ovular, or triangular cross section. FDMs may be comprised of a single component, or a series of components, for example a series of spherical components (e.g., ovoid, elliptical, or spherically-shaped beads or “pearls”). FDMs may be of any suitable material. For example, the FDMs may be made of nitinol, polymer, plastic, polyester, or metal. Further, the surface of the FDM may be textured and/or be coated. For example, the surface of the FDM may have a pattern of cutouts and/or ridges, which may help facilitate integration with cardiac tissue. Optionally, a FDM may comprise a coating or fabric that may help induce tissue formation and incorporation such that shortly after implantation, the implant may become at least partially incorporated into the wall of the left ventricle (LV). Further, one or more portions of a FDM may comprise a radiopaque material, such as barium sulfate. The radiopaque material may be distributed throughout the FDM (240) and/or may be concentrated at particular regions or bands on the FDM, as may be desirable. This may provide visibility of the force distribution members while they are being implanted, which may aid the implantation of the implantable device at the desired location and/or orientation. It may be beneficial for a user to be able to see the force distribution members while the implantable device is being implanted so that the user can see the location of the implantable device within the ventricle, and ensure the implantable device is properly situated, for example. A FDM may be made entirely of bioabsorbable or biodegradable materials, entirely of non-bioabsorbable or non-biodegradable materials, or may be a composite structure where some portions are bioabsorbable or biodegradable and some portions are not.

Although the devices for deployment have been described above as an implants including a plurality of anchors coupled to a tether, it is understood that other catheter-based devices may be deployed from the systems. For example, various types of clip or staple devices may be deployed for attaching a prosthetic valve, repairing a cardiac valve, closing a left atrial appendage, closing an atrial septal defect, or closing a ventricular septal defect.

### Guide Catheter and Multi-Window Catheter

The systems described herein may also include one or more of a guide catheter and a multi-window catheter (e.g., a template catheter). The guide catheter may be any catheter configured for advancement within the cardiovascular anatomy to one or more target sites of a tissue, e.g., a cardiac tissue. The guide catheter will typically have a length and diameter that allows one or more additional catheters to be advanced and/or retracted therethrough. For example, a multi-window catheter may be coaxially disposed within a lumen of the guide catheter, and advanced within the lumen to one or more tissue sites. FIGS. 12A and 12B depict one variation of a multi-window catheter (400) comprising an outer catheter (410) having a lumen (414) and a series of openings (412) in and along a sidewall (416) of the outer catheter (410). The placement of the openings along multi-window catheter may aid in securing the anchors in a desired configuration. For example, the spacing or distance between each opening may correspond to the desired spacing between anchors. In some variations, the spacing or distance between anchors (and the corresponding spacing or distance between each opening of the multi-window catheter) may be from about 6 mm to about 20 mm, e.g., from about 6 mm to about 12 mm, from about 8 mm to about 13 mm, from about 10 mm to about 15 mm, from about 15 mm to about 18 mm, from about 12 mm to about 20 mm, about 10 mm, about 11 mm, about 11.5 mm, about 12.5 mm, etc.). The number of openings (412) may correspond to the desired number of anchors to be implanted, though the number of anchors delivered can be greater (if more than one anchor is deployed from a window) or fewer than the number of openings. In some variations, a multi-window catheter may comprise an inner catheter that is slidable within the outer catheter. For example, FIG. 12B depicts a multi-window catheter comprising an outer catheter (410) and an inner catheter (450) slidable within the outer catheter (410). The inner catheter (450) may comprise a lumen and a sidewall opening, wherein aligning the side wall opening of the inner catheter with each of the sidewall openings of the multi-window catheter helps guide the sequential delivery of individual anchors through each of the sidewall openings to target anchor locations. Each of the anchors of the implantable device may be implanted into the ventricle wall at a pre-selected depth by deploying anchors through the openings in the inner and outer multi-window catheter when it is located at a pre-selected depth within the ventricle.

### Methods

Methods for synchronizing the deployment of various devices into tissue of a patient, e.g., a heart tissue, with an ECG waveform from the patient are also described herein. In general, the methods may include obtaining an ECG signal from a patient, synchronizing deployment of the device from a deployment catheter with a portion of the ECG signal, and deploying the device into one or more target sites of a cardiac or cardiovascular tissue. The one or more target sites may include an area of reduced tissue thickness. In some variations, the area of reduced thickness may be about 4 mm or less. This amount of reduced thickness may be the thickness of the cardiac tissue during a diastolic portion of a cardiac cycle. The methods may be used to treat various medical conditions, but may be generally be used to treat heart failure due to any etiology, e.g., hypertension, myocardial infarction, valvular insufficiency, and/or a congenital defect.

Deployment of a device may be synchronized with a portion of the ECG signal. In some variations, the portion of the ECG signal may correspond to an end systolic portion of a cardiac cycle. In other variations, the portion of the ECG signal may correspond to a QRS complex of the ECG signal. In further variations, deployment of a device may be synchronized with an ST segment of the ECG signal. Synchronizing deployment may be accomplished using one or more processors configured to analyze the ECG signal and then automatically issue a control signal to the deployment catheter that may trigger deployment of the device. In some variations of the methods, the one or more processors may obtain and analyze an ECG signal, but may also include a step whereby deployment of the device may be at least partially manually controlled by a user. In some instances, delivering a device into one or more target sites comprises use of a pressurized gas. The pressurized gas may include oxygen, helium, nitrous oxide, nitrogen, medical air, carbon dioxide, hydrogen, or mixtures thereof. The methods described herein may further include showing on a display of the system, an ECG signal, one or more physiological signals (e.g., heart rate, respiratory rate, blood pressure), an image of an anatomical area of the patient, real-time deployment of a device, or a combination thereof.

For example, as shown in FIG. 1A, a region of myocardial tissue (1) is shown in diastole with a sufficient thickness of about 5 mm to about 6 mm or more. There is a reduced tissue thickness zone (2), which has a first thickness (3) that is about 4 mm or less, which may be less than desired for safe delivery of a device, and which may lead to perforation. At systole, as shown in FIG. 1B, thin-walled region (2) has expanded to a second, greater thickness (4) of about 5 mm to about 6 mm or more, which may be sufficient to safely deliver a device and to avoid perforation.

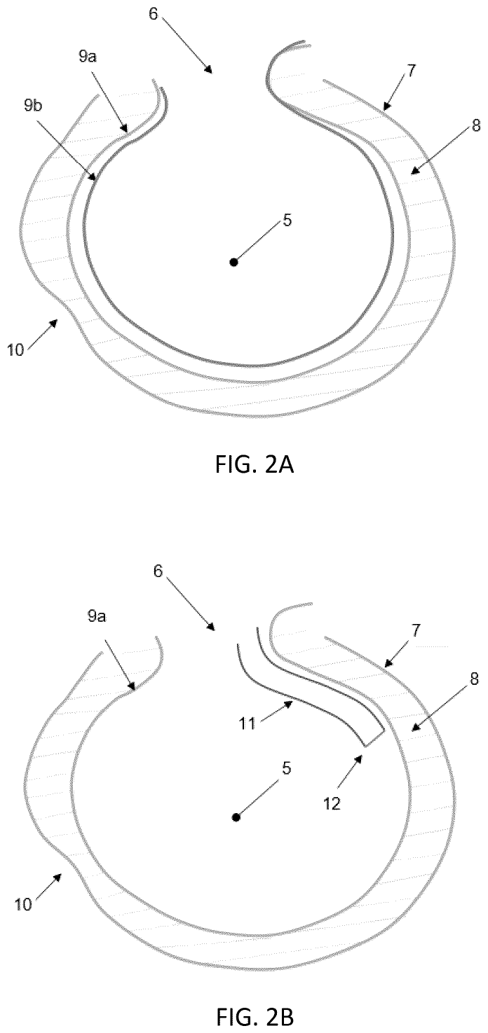

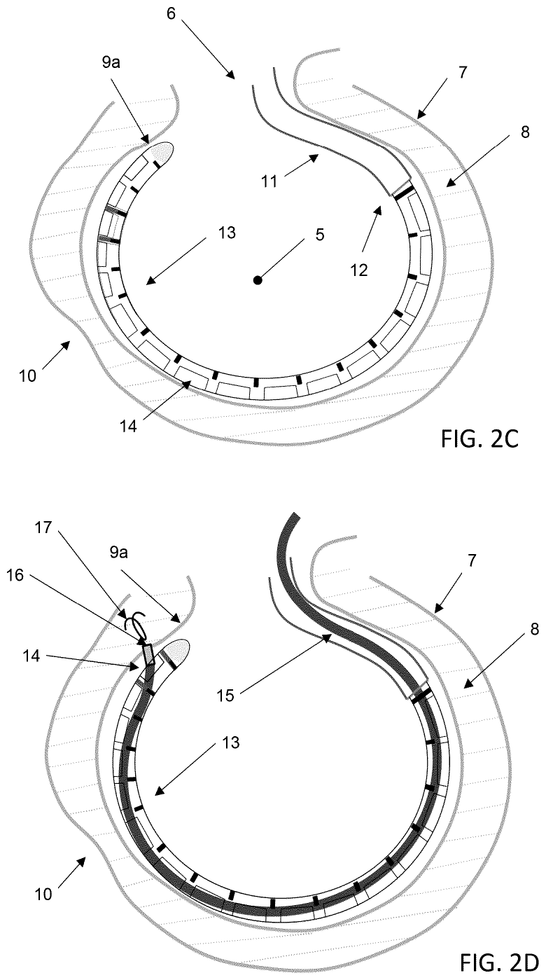

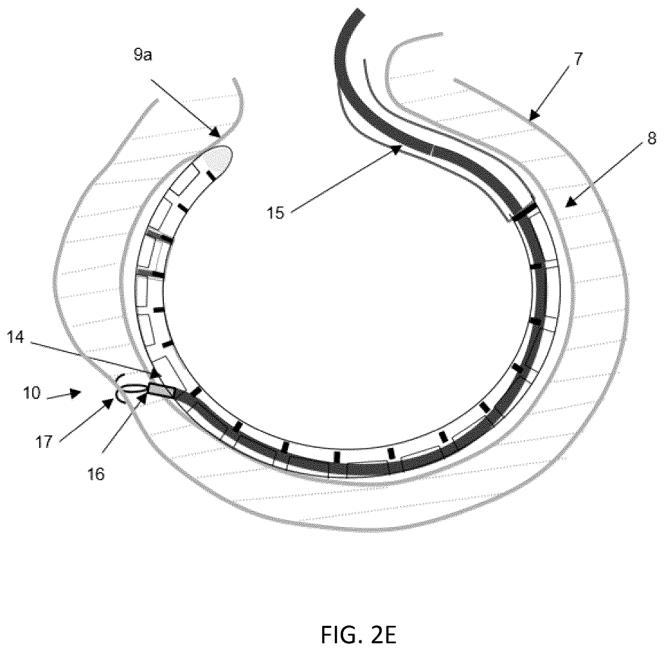

An example of delivering devices into the myocardium of the left ventricle (LV) with reduced ejection fraction heart failure (HFrEF) is shown starting in FIG. 2A, which depicts a short axis (SAX) view of the LV at the end of diastole. HFrEF patients have an LV end diastolic diameter (LVEDD) greater than about 55 mm, and often between about 65 and about 85 mm or more. Also shown are the LV cavity (5), left ventricular outflow tract (LVOT) (6), epicardium (7), myocardium (8), endocardium (9a), and a thin-walled region (10). In healthy subjects, myocardium (8) may be about 10 mm or more in thickness at the end of diastole. In HFrEF patients the LV diameter generally increases and the wall thins, such that myocardium (8) may be reduced to about 5 mm to about 8 mm in thickness, while thin-walled region (10) may be as little as about 2 mm to about 4 mm thick. Throughout the cardiac cycle, epicardium (7) generally remains stationary, while the myocardium (8) thickens due to myofiber shortening. This thickening typically manifests in a radially inwardly directed endocardial wall motion, as represented by the change from end diastolic position (9a) to end systolic position (9b). HFrEF patients typically have systolic dysfunction, exhibited by reduced contractility and wall thickening during systole. The distance traveled by endocardium (9a) from the end of diastole to its end-systolic location (9b) may be less than in the healthy population, and may range from about 2 mm to about 6 mm or about 7 mm, such that in the HFrEF LV at the end of systole, the myocardium (8) may increase in thickness from about 7 mm to about 14 mm or 15 mm, while at thin-walled region (10), the wall may increase in thickness from about 4 mm to about 11 mm, including all values and sub-ranges therein.

In general, to gain access to the LV, as shown in FIG. 2B, a guide catheter (11) may be inserted through the LVOT (6). The guide catheter (11) may include a distal tip (12) and be configured to sit against the endocardium (9a). A multi-window catheter (12) may be placed and advanced through the guide catheter (11) such that distal tip (12) may be tracked around the endocardium (9a) to span substantially most of the LV perimeter. Multiple windows (14) may be included along the outer radius of the multi-window catheter (13), through which, as illustrated in FIG. 2D, a deployment catheter (15) may exit to contact or penetrate the endocardium (9a) with its distal tip (16) once it is tracked up and through the multi-window catheter (13). Once the distal tip (16) has entered into the myocardium (8), a device (17) may be delivered, and due to the local myocardial thickness, the device (17) may reside within the myocardium (8) without penetrating the epicardium (7).

When a region of the myocardium includes a thin wall (10), as shown in FIG. 2E, a properly placed deployment catheter (15) and distal tip (16) exiting from a window (14) near thin-wall region (10) may perforate the epicardium (7). In either region, triggering deployment of the device may occur while the system is in an automated mode, in which deployment may be automated with the assistance of one or more processors configured to synchronize deployment with a target portion of the ECG signal. However, switching to the automated mode from a manual mode may be controlled by the operator.

As previously mentioned, to overcome the potential to perforate the epicardium while deploying a device, methods that synchronize deployment with the ECG signal (e.g., ECG waveform) may be leveraged. Referring to FIG. 3, a typical ECG signal is depicted highlighting the QRS complex (18a) and the T wave (18b), which represents ventricular depolarization and systolic contraction, and repolarization and diastolic relaxation, respectively. Typically, the myocardium thickens during systole and thins during diastole. Thus, methods that leverage the ECG waveform may employ a two-step deployment sequence: first, the operator may enable actuation of a deployment catheter once proper location against the endocardium or penetration into the myocardium is achieved. Second, the method may trigger the system to fire or deploy the device upon receiving a control signal that synchronizes deployment with a target portion of the ECG waveform that corresponds to the end of the systolic portion of the cardiac cycle, e.g., the QRS complex. Such a system was illustrated in FIG. 4, in which an ECG monitor (19), electrically connected to a controller (20), which accepts and interprets the ECG waveform was depicted. As previously mentioned, a deployment catheter (21) may also be connected to the controller (20). In use, an operator may advance the shaft (25) of the deployment catheter (21) through a multi-window catheter (shown in FIG. 12), which would bring it into proximity of one or more target sites of the target tissue. The operator would then grasp the deployment catheter (21) by its handle (22), to advance the deployment catheter (21) to the proper location against or into the myocardium. Once the proper position has been achieved, the operator may actuate an enabling switch (23), which may then send an enabling signal to controller (20). Upon receiving the next systolic QRS complex, the controller (20) may send a control signal to an actuator (24), which may be attached to a portion of the handle (22). Actuation of actuator (24) may then deploy a delivery device (27), depicted in detail (28), through the distal tip (26) of the deployment catheter (21) and into a target site of the myocardium.

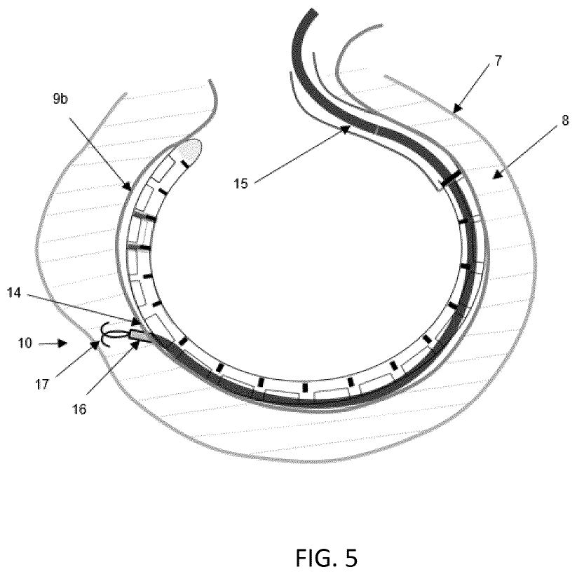

The system is shown in use in FIG. 5, which depicts the same epicardium (7) and myocardium (8) as shown in FIG. 2E, but with the cardiac cycle at the end of systole such that the radial excursion of the endocardium (9b) presents a thicker myocardium at the thin-walled region (10). When the device (17) is synchronized for deployment at the end of systole, deployment may be completely within myocardium (8), without violating epicardium (7).

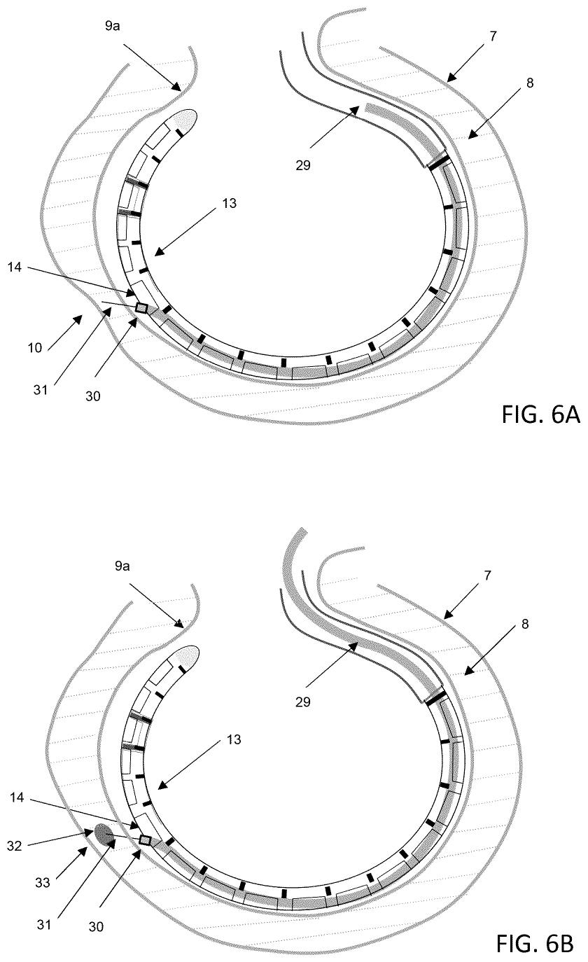

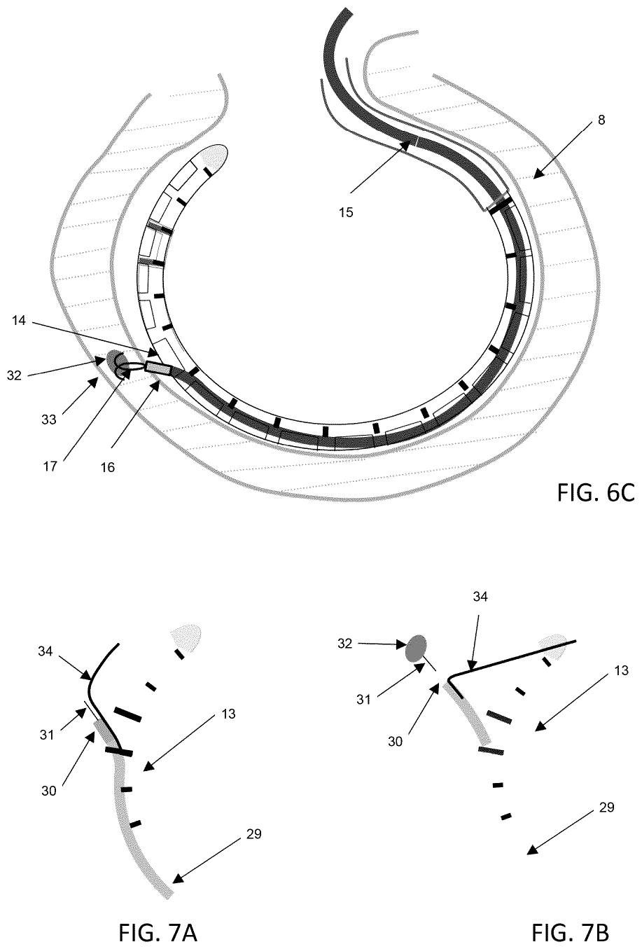

In certain disease states, the thin-walled myocardium (10) of FIG. 5 may not thicken at any point throughout the cardiac cycle. In such disease states the tissue may first be augmented by delivering a substance into the myocardium (8) at or near the region of thin-walled region (10) to thicken it prior to deploying the device (17). For example, as illustrated in FIG. 6A, an ancillary catheter, e.g., a substance catheter (29), is shown with a distal tip (30) and cannula (31) advanced through a multi-window catheter (13) and out window (14) such that the cannula (31) enters the myocardium (8) near thin-walled region (10). A substance (32), which may be radiopaque, may then be injected (FIG. 6B) into thin-walled region (10), to create an augmented (e.g., increased) wall thickness (33), with sufficient thickness to deliver device (17) (FIG. 6C) into the augmented region (33), with or without use of the synchronized system of FIG. 4, and without perforation.

Cardiac catheterization laboratory procedures involving a patient's LV typically employ fluoroscopy for visualization, with the fluoroscopic C-arm oriented to visualize the LV from the SAX view. Given that soft tissues such as myocardium and fat are radiolucent, radiopaque markers and materials may be used to help navigate the system components and devices described herein. For example, FIG. 7A shows a multi-window catheter (13) and substance catheter (29) as they might appear on fluoroscopy, with a distal tip (30) and cannula (31) visualized. Given cardiac tissue is radiolucent, to visualize the location of the endocardium a radiopaque member such as an indicator wire (34) may be used, which by its curved region may show the endocardial border. Once the substance catheter (29) is advanced into the myocardium such that the cannula (31) is at the appropriate depth, the substance (32), which is radiopaque in this example and can be made of many candidate materials or combination of materials, may be delivered to augment (e.g., increase) the thickness of the myocardium (FIG. 7B). One such material may comprise a double network hydrogel (DN hydrogel). Exemplary DN hydrogels may include a first network of Poly(ethylene glycol) methyl ether methacrylate and a second network of acrylic acid. DN hydrogels may exhibit greater toughness than conventional hydrogels, although conventional hydrogels may also be employed. Radiopaque doping may be added to the substance (32) to facilitate visualization on fluoroscopy during and after deployment.

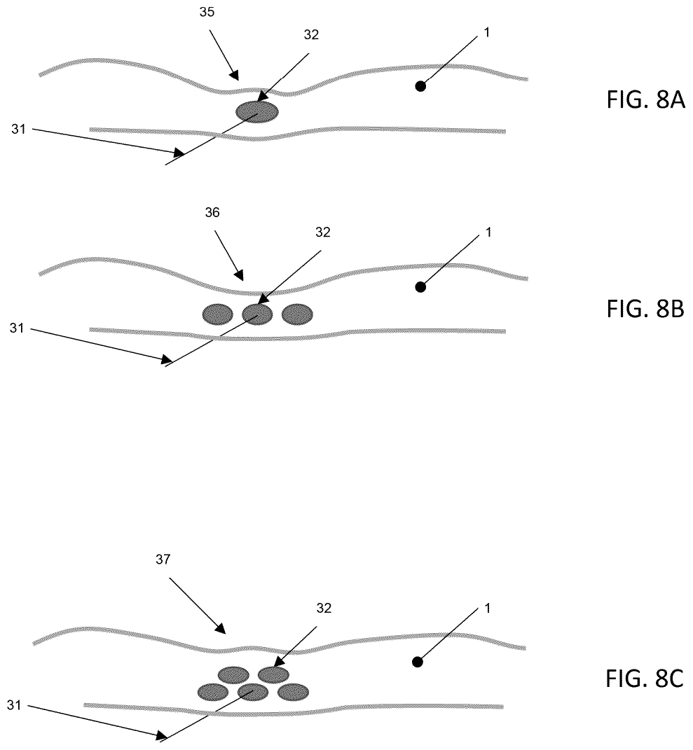

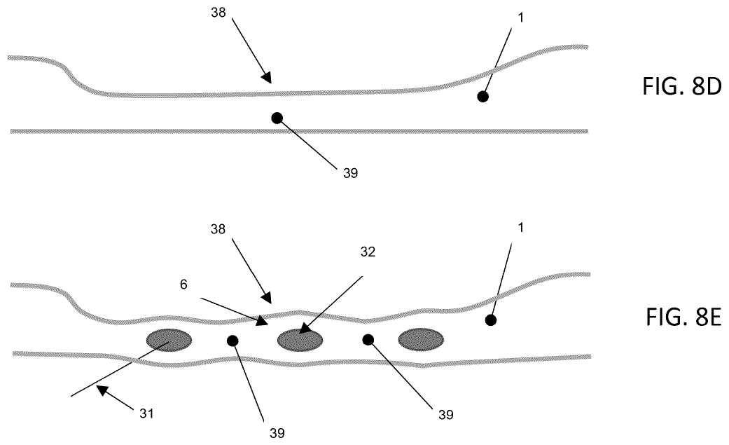

Tissue augmentation may be accomplished using varying techniques to inject or distribute the substance (32). For example, as shown in FIG. 8A, a single bolus of substance (32) may be injected through the cannula (31) to locally augment (e.g., increase thickness of) a thin region (35) of tissue (1). Alternatively, as illustrated in FIG. 8B, multiple boluses of substance (32) may be injected in series to augment a broader region (36) of tissue. Another alternative may be to deliver boluses of substance (32) in series and in parallel, as depicted in FIG. 8C, to create a broad region (37) of substantially thicker tissue.

In disease states where a thin-wall region (38) with a first thickness (39) spans a substantial portion of the LV perimeter (FIG. 8D), and in which multiple devices are to be implanted, the substance (32) may be injected over that span (FIG. 8E) to locally augment tissue to a second, greater thickness at specific regions of the tissue, while leaving other regions (39) in which no device deployment is planned without augmentation.

### Reshaping Tissue

After deployment, the devices described herein may be used to reshape a heart ventricle. In some variations, reshaping a heart ventricle may comprise implanting a device including a plurality of anchors coupled to a tether into ventricular wall tissue approximately 10-15 mm below a mitral valve plane, and cinching the device from an uncinched configuration to a cinched configuration. Alternatively or additionally, reshaping may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion. The reshaping methods may further comprise securing the device in a cinched configuration. It is understood that implantable device need not comprise a plurality of anchors coupled to a tether and may be any device that is configured to cinch, tighten, shrink, tension, plicate, and/or otherwise draw tissue together.

One variation of an implantable device that may be used with any of the methods described herein may comprise a sleeve or sheath coupled to one or more helical tissue-penetrating members which may be secured into the ventricular wall of a heart. Helical tissue-penetrating members may be, for example, prongs or screws having external helical threads, helical fasteners (e.g., corkscrew-like fasteners) having a pointed tissue-piercing tip, and the like. The sheath may be comprised of flexible material. The sheath may further comprise a wire or thread “spine” threaded through the sleeve such that tensioning the spine cinches the device. To affix the sheath or sleeve to the ventricular wall, the helical tissue-penetrating members may be located within or partially within the sleeve or sheath such that the tissue-penetrating members penetrate through the sleeve or sheath into the ventricular wall tissue, or the helical tissue-penetrating members may be fixedly attached to a surface of the sheath. A knot and/or lock member may be secured to the spine in order to maintain the device in a cinched configuration. The spine may be tensioned in any suitable manner. For example, a catheter may be configured to rotate the lock member to tension the spine. The device may comprise one or more radiopaque components to aid in visualization during implantation.

In another variation, an implantable device may comprise a flexible sleeve and one or more sutures coupled to the sleeve. The sutures may be slidably threaded through the sleeve, and through the ventricular wall to attach the sleeve to the ventricle. Tensioning a suture that has been threaded throughout the sleeve and into and out of ventricular wall tissue may cinch the implant and the ventricular wall tissue.

Some variations of implantable devices may be self-cinching. For example, an implantable device may be made of shape-memory material such that the device can be implanted in a restrained, uncinched configuration, and revert to a cinched configuration when it is no longer restrained or under tension. For example, an implantable device may comprise a portion made of shape memory material coupled to attachment mechanisms such as anchors, sutures, or helical tissue penetrating members. The attachment mechanisms may secure the shape memory portion of the device to the ventricular wall. In some variations, the attachment mechanisms may be secured to the tissue while the device is in an uncinched configuration (e.g. under tension), and the device may be released from tension or unrestrained such that it reverts back to a cinched configuration, thereby applying force to reduce the circumference of the ventricular wall at the location of the device. For example, a shape memory device may be restrained in an uncinched configuration by a delivery catheter, and released from the catheter after implantation into ventricular tissue, allowing the device to revert to a cinched configuration.

In another variation, an implantable device made of shape memory material may be placed under tension as it is implanted. Once fully implanted, the tendency of the device to revert back to an uncinched configuration may apply a cinching force to the ventricular wall. Such a device would not require a separate step to cinch the device, but would automatically assume the cinched configuration once unrestrained. Any of the device and methods described herein may be used to implant the device in the desired location and desired orientation, and cinch and lock the device by a desired amount. Alternatively, other methods and/or device may be used to implant, lock, and cinch an implantable device.

As described above, methods for reshaping (e.g., reverse-remodeling) a heart ventricle may comprise securing an implantable, cinchable device at a location about 10-15 mm below the mitral valve, and optionally in a plane approximately parallel to the plane of the mitral valve (e.g., a plane defined by the mitral valve annulus or a plane defined by one or more of the mitral valve leaflets). The device may also be implanted at an angle a1 to the mitral valve plane, and in some variations, may be longer (e.g., has more tissue anchors and FDMs, has a greater length between the distal anchor and proximal anchor) than a device that is implanted at an angle a2 to the mitral valve plane, where angle a2 is less than angle a1 (e.g., a device that is implanted approximately parallel to the mitral valve plane). Securing the implantable device (200) may comprise securing a plurality of tethered anchors into the ventricle wall. Securing an implantable device into the ventricle wall may comprise using various catheters to position and secure the plurality of anchors to a location within the ventricle. For example, securing an implantable device to the ventricle may comprise using catheters to position and secure one or more anchors at a location approximately 10-15 mm below the mitral valve. Some methods may comprise securing the implantable device into ventricular wall tissue that is from about 3 mm to about 25 mm below a mitral valve plane (e.g., from about 7 mm to about 20 mm, from about 10 mm to about 15 mm), for example, securing the implantable device in myocardium bounded by the mitral valve plane (and/or the subannular groove) and papillary muscle insertion.

In some variations, a multi-window catheter may be configured to facilitate the delivery of the implantable device at the desired or pre-determined location below the mitral valve. A multi-window catheter may comprise a distal portion having a pre-defined curvature that approximates the radius of curvature of the ventricle approximately 10-15 mm below the mitral valve and/or that approximates the radius of curvature of the widest portion of the ventricle. Optionally, the distal portion of the multi-window catheter may be stiffened relative to its proximal portion so that the distal portion retains its curvature in a beating heart procedure, which may facilitate positioning or seating the multi-window catheter along the ventricular wall approximately 10-15 mm apical to the mitral valve. A stiffened distal portion that has a radius of curvature that approximates, or is larger than, the radius of curvature of the ventricle at or around the desired implantation location may help the multi-window catheter seat itself at or around the implantation location while the heart is beating. As described above, securing a cinchable implantable device approximately 10-15 mm below the mitral valve annulus may facilitate therapeutic remodeling of a heart ventricle as compared to methods that secure a cinchable implantable device at locations closer to the mitral valve (e.g., at the mitral valve annulus).

In some variations, the method of securing an implantable device to the ventricle may comprise positioning a multi-window catheter in the ventricle to deliver the implantable device at a pre-selected location in the ventricle. For example, securing the implantable device to the ventricle may comprise advancing a guide catheter to ventricular tissue at or near the mitral valve (e.g., at or near the mitral valve annulus and/or subannular groove region in the left ventricle, along the anterolateral wall and in the sub-valvular space behind chordae tendineae) and advancing the multi-window catheter through the guide catheter, and positioning the multi-window catheter at a location below the mitral valve plane (e.g., from about 3 mm to about 25 mm below the mitral valve plane, from about 7 mm to about 20 mm below the mitral valve plane, from about 10 mm to about 15 mm below the mitral valve plane, in myocardium bounded by the mitral valve plane and papillary muscle insertion).

The multi-window catheter may also facilitate repeatable delivery of anchors at target implant sites along the ventricular wall. For example, the plurality of openings of the outer catheter may permit delivery of tissue anchors with pre-determined spacing and/or alignment with respect to each other. The number and spacing of the openings of in the multi-window catheter may also dictate the span of the plurality of anchors across the ventricle wall. Turning to FIGS. 12A and 12B, the multi-window catheter (400) may comprise an outer catheter (410) comprising a plurality of openings (412) and a lumen (414), and an inner catheter (450) that is slidable within the lumen (414) of the outer catheter. The inner catheter may facilitate the placement of an anchor at a target anchor location by directing an anchor delivery catheter through a single side wall opening of the outer catheter while restricting or blocking access through the other side wall openings. Methods herein may comprise sliding the inner catheter (450) within the outer catheter (410) to reveal an unobstructed opening. For example, the inner catheter (450) may comprise an opening and may be slidable within the outer catheter. Thus, the inner catheter (450) can be aligned within the outer catheter (410) such that only one opening is left unobstructed. By aligning the inner catheter (450) with the outer catheter (410) in a particular fashion, only one opening will be available for the anchor delivery catheter to extend through and secure an anchor to a ventricle wall. Thus, the multi-window catheter (400) may help reduce the likelihood that an anchor is deployed to an incorrect or undesired location. However, the inner catheter (450) may have any suitable number of openings. For example, it may be desirable to allow two openings in the sidewall of the outer catheter to remain unobstructed. Thus, the inner catheter may comprise two openings spaced apart to correspond with the openings in the outer catheter.

As described above, methods of securing an implantable device to the ventricle wall may comprise positioning anchors at pre-determined locations in the ventricle and at a pre-selected distance apart from each other. The outer catheter (410) of the multi-window catheter system (400) may facilitate placement of the anchors at a pre-selected distance apart from each other. The outer catheter (410) may comprise any suitable number of openings (412), spaced apart by any suitable distance (418), as depicted in FIG. 12A. In one variation, the multi-window catheter may comprise 11 to 16 openings. Other variations may have about 5-10 openings while still other variations may have about 17-25 openings. Any suitable number of anchors may be delivered through each opening (412). In some variations, the method may comprise delivering one anchor through each opening. Thus, the number of openings in the outer catheter may correspond to the number of anchors secured in the ventricle. For example, in a variation where the multi-window catheter comprises 11 to 16 openings, 11 to 16 anchors may be delivered (one per opening). The distance between a first and last opening of the multi-window catheter may correspond to the approximate length of the implantable device when it is implanted (but before it is cinched). When one anchor is deployed through each opening in the multi-window catheter, the location and spacing of each anchor is determined by the spacing of the openings of the multi-window catheter. The openings (412) of the multi-window catheter (400) may be configured to facilitate the implantation a device that spans any suitable distance.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A system for deploying a device into tissue comprising:
a) a deployment catheter; and
b) a controller comprising one or more processors, wherein the one or more processors is configured to analyze an ECG signal from a patient and issue a control signal to the deployment catheter to deliver the device into one or more target sites of a cardiac tissue, wherein the one or more target sites comprises an area of reduced tissue thickness.

2. The system of claim 1, wherein the area of reduced tissue thickness is about 4 mm or less during a diastolic portion of a cardiac cycle.

3. The system of claim 1, wherein the control signal is operable to trigger the deployment catheter to deliver the device therefrom during a target portion of the ECG signal.

4. The system of claim 3, wherein the target portion of the ECG signal corresponds to an end systolic portion of a cardiac cycle.

5. The system of claim 3, wherein the target portion of the ECG signal comprises a QRS complex.

6. The system of claim 3, wherein the target portion of the ECG signal comprises an ST segment.

7. The system of claim 1, wherein the cardiac tissue comprises a left ventricular tissue.

8. (canceled)

9. The system of claim 1, wherein the cardiac tissue comprises myocardium.

10. The system of claim 1, wherein the deployment catheter comprises:
a) an elongate shaft;
b) a slider configured to translate along a longitudinal axis of the shaft; and
c) a handle coupled to a proximal end of the shaft and comprising an actuator coupled to a body of the handle, wherein the actuator is configured to deliver the device from a lumen of the catheter according to the control signal.

11. The system of claim 10, wherein the actuator comprises a cylinder configured to be filled with a pressurized gas selected from the group consisting of oxygen, helium, nitrous oxide, nitrogen, medical air, carbon dioxide, hydrogen, and mixtures thereof.

12. (canceled)

13. The system of claim 10, wherein the actuator comprises a solenoid valve.

14. (canceled)

15. (canceled)

16. The system of claim 1, wherein the device comprises an implant including a plurality of anchors coupled to a tether.

17. (canceled)

18. The system of claim 16, wherein the implant further comprises a plurality of force distribution members.

19.-21. (canceled)

22. The system of claim 1, further comprising a multi-window catheter.

23. (canceled)

24. The system of claim 1, further comprising a display, the display comprising a manual interface operable by a user to switch the one or more processors to a manual mode in which the user manually controls delivery of the device from the deployment catheter.

25.-29. (canceled)

30. A method for deploying a device into tissue comprising:
a) obtaining an ECG signal from a patient;
b) synchronizing deployment of the device from a deployment catheter with a target portion of the ECG signal; and
c) deploying the device into one or more target sites of a cardiac tissue, wherein the one or more target sites comprise an area of reduced tissue thickness.

31. The method of claim 30, wherein the area of reduced tissue thickness is about 4 mm or less during a diastolic portion of a cardiac cycle.

32. The method of claim 30, wherein the target portion of the ECG signal corresponds to an end systolic portion of a cardiac cycle.

33. The method of claim 30, wherein deployment of the device is synchronized with a QRS complex of the ECG signal.

34. The method of claim 30, wherein deployment of the device is synchronized with an ST segment of the ECG signal.

35. The method of claim 30, wherein synchronizing deployment is accomplished using one or more processors configured to analyze the ECG signal and issue a control signal to an actuator of the deployment catheter such that the device is deployed into the one or more target sites during the target portion of the ECG signal.

36. The method of claim 30, wherein the cardiac tissue comprises a left ventricular tissue.

37. (canceled)

38. The method of claim 30, wherein the cardiac tissue comprises myocardium.

39. The method of claim 30, wherein deploying the device into the one or more target sites comprises actuating a flow of a pressurized gas selected from the group consisting of oxygen, helium, nitrous oxide, nitrogen, medical air, carbon dioxide, hydrogen, and mixtures thereof.

40. (canceled)

41. The method of claim 30, further comprising reshaping the left ventricular tissue with the device.

42. (canceled)

43. The method of claim 30, wherein the device comprises an implant including a plurality of anchors coupled to a tether.

44. (canceled)

45. The method of claim 43, wherein the implant further comprises a plurality of force distribution members.

46.-50. (canceled)

51. The method of claim 43, wherein the deploying the device comprises deploying the plurality of anchors into the cardiac tissue sequentially.

52. The method of claim 43, wherein the deploying the device comprises deploying the plurality of anchors into the cardiac tissue simultaneously.

53. (canceled)

54. The method of claim 30, further comprising switching from an automatic mode to a manual mode in which the user manually controls deployment of the device from the deployment catheter.

55.-59. (canceled)
